(19) United States
(12) Patent Application Publication
(10) Pub. No.: US 2007/0071643 A1
(43) Pub. Date: Mar. 29, 2007

(54) Internet based system for monitoring blood test, vital sign and exercise information from a patient

(75) Inventors: Christopher Hall, San Francisco, CA (US); Vance Whitson Lanier JR., San Francisco, CA (US); David Shewmake, San Francisco, CA (US); Frank Ruderman, San Rafael, CA (US); Matthew Banet, Del Mar, CA (US); Randon Schultz, Venice, CA (US); Marshal Dhillon, San Diego, CA (US); Adam Fleming, San Diego, CA (US); Henk Visser, San Diego, CA (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: Berkeley HeartLab, Inc.

(21) Appl. No.: 11/522,589

(22) Filed: Sep. 18, 2006

Related U.S. Application Data
(60) Provisional application No. 60/721,617, filed on Sep. 29, 2005.

(51) Int. Cl. G01N 33/00

(52) U.S. Cl. 422/62; 422/67

(57) ABSTRACT

The invention provides a system for monitoring a patient that includes: 1) a first database that stores the patient's blood test information; 2) a monitoring device that collects the patient's cardiovascular and exercise information; 3) a second database that receives cardiovascular and exercise information from the monitoring device; and 4) an Internet-based system that displays the blood test, cardiovascular, and exercise information.

### CROSS REFERENCES TO RELATED APPLICATION

This application claims the benefit of U.S. Provisional Application Ser. No. 60/721,617 filed Sep. 29, 2005 and is hereby incorporated by reference.

### BACKGROUND

1. Field of the Invention

The present invention relates to patient-monitoring systems that process and analyze: i) information collected from one or more blood tests; ii) vital sign information, such as heart rate, pulse oximetry, blood pressure, and weight, collected from a vital sign monitor; and iii) exercise information, such as steps, duration of exercise, and calories burned, collected from an exercise monitor.

2. Description of the Related Art

Although mortality rates for cardiovascular disease (CVD) have been declining in recent years, this condition remains the primary cause of death and disability in the United States for both men and women. Currently CVD affects approximately 12 million Americans. Atherosclerotic cardiovascular disease (ASCVD), a form of CVD, can cause hardening and narrowing of the arteries, which in turn restricts blood flow and impedes delivery of vital oxygen and nutrients to the heart. This can lead to coronary heart, cerebral vascular, and peripheral vascular diseases, and results in approximately 75% of all deaths attributed to CVD.

Elevated concentrations of low-density lipoprotein cholesterol (LDL cholesterol) are causally related to the onset of ASCVD because over time these compounds contribute to a harmful formation of plaque on an artery's inner walls, thereby restricting blood flow. The likelihood a patient has ASCVD increases with their concentration of LDL cholesterol, which is therefore typically referred to as ‘bad cholesterol’. Conversely, high-density lipoprotein cholesterol (HDL cholesterol) can bind with LDL cholesterol in the bloodstream and transport it to the liver for disposal. Because of this process, called ‘reverse cholesterol transport’, a high level of HDL cholesterol appears to lower a patient's risk of developing heart disease and stroke. HDL cholesterol is therefore typically referred to as ‘good cholesterol’.

A lipoprotein analysis (also called a lipoprotein profile or lipid panel) is a blood test that measures, among other compounds, blood levels of total cholesterol, LDL cholesterol, and HDL cholesterol. One method for measuring HDL and LDL cholesterol is described in U.S. Pat. No. 6,812,033, entitled ‘Method for identifying risk cardiovascular disease patients’. This patent, assigned to Berkeley HeartLab Inc. and incorporated herein by reference, describes a blood test based on a gradient-gel electrophoresis (GGE). Gradient gels used in GGE are typically prepared with varying concentrations of acrylamide and can separate macromolecules with relatively high resolution compared to conventional electrophoretic gels. Sub-classes of both HDL and LDL cholesterol can be determined by GGE. For example, GGE can differentiate up to seven subclasses of LDL cholesterol (classified as LDL I, IIa, Ilb, Illa, Illb, IVa, and IVb), and up to five subclasses of HDL (classified as HDL 2b, 2a, 3a, 3b, 3c). These tests correlate to a technique called analytic ultracentrifugation (AnUC), which is an established clinical research standard for lipoprotein subfractionation.

GGE can differentiate the most atherogenic particles, LDL IIIa, IIIb, and IVb, and also the most helpful HDL particle, HDL 2b. Elevated levels of LDL IVb, which represents the smallest LDL cholesterol particles, have been reported to have an independent association with arteriographic progression; a combined distribution of LDL IIIa and LDL IIIb typically reflects the severity of this trait. High levels of HDL 2b increase the efficacy of reverse cholesterol transport, while low levels of HDL 2b can increase the risk of CVD.

GGE can be combined with other blood tests to collectively measure the following compounds:

Total Cholesterol

LDL Cholesterol (and subclass distribution)

HDL Cholesterol (and subclass distribution)

Triglycerides

Apo B-Particle

Apo B Ultra Particle

Lipoprotein

Apo E Genotype

Fibrinogen

Folate

HbA_{1c}

C-Reactive Protein

Homocysteine

Glucose

Insulin

Chlamydia

Other Compounds

Elevated blood pressure is another significant risk factor for CVD. The relationship between blood pressure and the risk of CVD is typically continuous, consistent and independent of other risk factors. For example, each increment of 20 mmHg for systolic blood pressure and 10 mmHg for diastolic blood pressure doubles the risk of CVD across the entire blood pressure range, starting with a pressure of 115/75 mm Hg. Lifestyle modifications, such as weight loss, diets that reduce sodium and fat, smoking cessation, increase in aerobic activity, and reduction in alcohol intake, can lower blood pressure, and thus reduce the risk of CVD.

A medical device called a sphygmomanometer measures a patient's blood pressure using an inflatable cuff and a sensor (e.g., a stethoscope) that detects blood flow by listening for sounds called the Korotkoff sounds. During a measurement, a medical professional typically places the cuff around the patient's arm and inflates it to a pressure that exceeds their systolic blood pressure. The medical professional then incrementally reduces pressure in the cuff while listening for flowing blood with the stethoscope. The pressure value at which blood first begins to flow past the deflating cuff, indicated by a Korotkoff sound, is the systolic pressure. The stethoscope monitors this pressure by detecting strong, periodic acoustic ‘beats’ or ‘taps’ indicating that the blood is flowing past the cuff (i.e., the systolic pressure barely exceeds the cuff pressure). The minimum pressure in the cuff that restricts blood flow, as detected by the stethoscope, is the diastolic pressure. The stethoscope monitors this pressure by detecting another Korotkoff sound, in this case a ‘leveling off’ or disappearance in the acoustic magnitude of the periodic beats, indicating that the cuff no longer restricts blood flow (i.e., the diastolic pressure barely exceeds the cuff pressure).

Another medical device, called a pulse oximeter, measures a patient's blood oxygen content and heart rate. A typical pulse oximeter features an optical module, typically worn on a patient's finger or ear lobe, and a processing module that analyzes data generated by the optical module. The optical module typically includes first and second light sources (e.g., light-emitting diodes, or LEDs) that transmit optical radiation at, respectively, red (λ˜630-670 nm) and infrared (λ˜800-1200 nm) wavelengths. The optical module also features a photodetector that detects radiation transmitted or reflected by an underlying artery. Typically the red and infrared LEDs sequentially emit radiation that is partially absorbed by blood flowing in the artery. The photodetector is synchronized with the LEDs to detect transmitted or reflected radiation. In response, the photodetector generates a separate radiation-induced signal for each wavelength. The signal, called a plethysmograph, is an optical waveform that varies in a time-dependent manner as each heartbeat varies the volume of arterial blood, and hence the amount of transmitted or reflected radiation. A microprocessor in the pulse oximeter processes the relative absorption of red and infrared radiation to determine the oxygen saturation in the patient's blood. A number between 94%-100% is considered normal, while a value below 85% typically indicates the patient requires hospitalization. In addition, the microprocessor analyzes time-dependent features in the plethysmograph to determine the patient's heart rate.

### SUMMARY OF THE INVENTION

The present invention provides an Internet-based system that collects, analyzes, and displays the following for both patients and physicians, each of which is defined in more detail: 1) information from one or more blood tests; 2) vital sign information and exercise information collected by a monitoring device; and 3) personal information. Analysis of this information can yield a metabolic and cardiovascular risk profile that, in turn, can help the patient comply with a cardiovascular risk reduction program, and consequently manage their health. For example, using this information, the Internet-based system can generate a variety of personalized content, such as reports, recipes, reference articles, and recommendations for treatment, including recommendations for follow-on blood tests.

The Internet-based system can also include a bi-directional messaging system that sends personalized emails and text messages to the patient based on this information, as well as receives and processes messages sent from the patient. Messages typically include patient-specific content (e.g., treatment plans, diet recommendations, educational content) that helps drive the patient's compliance in a disease-management program (e.g. a cardiovascular risk reduction program), motivate the patient to meet predetermined goals and milestones, and encourage the patient to schedule follow-on medical appointments. Such a messaging system is described in a co-pending application entitled ‘INTERNET-BASED PATIENT-MONITORING SYSTEM FEATURING INTERACTIVE MESSAGING ENGINE’, filed Sep. 29, 2005, the contents of which are incorporated herein by reference.

Together, the Internet-based system, messaging engine, and monitoring device operate interactively and in concert to drive a constructive, personalized interaction between a medical professional and the patient. Ultimately these system form an effective tool that drives compliance and reduces risk that a patient's CVD progresses. ‘Blood test information’, as used herein, means information collected from one or more blood tests, such as a GGG-based test. Blood test information can include concentration, amounts, or any other information describing blood-borne compounds, including but not limited to total cholesterol, LDL cholesterol (and subclass distribution), HDL cholesterol (and subclass distribution), triglycerides, Apo B particle, Apo B ultra particle, lipoprotein, Apo E genotype, fibrinogen, folate, HbA_{1c}, C-reactive protein, homocysteine, glucose, insulin, chlamydia, and other compounds. ‘Vital sign information’, as used herein, means information collected from patient using a medical device, e.g. information that describes the patient's cardiovascular system. This information includes but is not limited to heart rate (measured at rest and during exercise), blood pressure (systolic, diastolic, and pulse pressure), blood pressure waveform, pulse oximetry, optical plethysmograph, electrical impedance plethysmograph, stroke volume, ECG and EKG, temperature, weight, percent body fat, and other properties. ‘Exercise information’, as used herein, means information that characterizes a patient's exercise habits, including but not limited to steps, miles run or biked, duration of exercise, degree of exertion during exercise, calories burned during exercise, and heart rate and other vital sign information measured during exercise. ‘Personal information’, as used herein, means information such as weight, age, gender, medical history, ethnicity, current medications, and other information that can be used in combination with the above-mentioned properties to, among other things, develop metabolic and cardiovascular risk profiles to diagnose and manage a patient.

Specifically, in one aspect, the invention provides a system for monitoring a patient that includes: 1) a first database that stores blood test information; 2) a monitoring device that collects the patient's vital sign and exercise information; 3) a second database that receives vital sign and exercise information from the monitoring device; and 4) an Internet-based system that displays information from the blood test, along with vital sign and exercise information. ‘Database’, as used herein, can mean a complete database (e.g., an Oracle® database) that includes a variety of data tables, or simply a single data table or portion of computer memory. For example, the first database can be part of a laboratory information management system (LIMS) that collects and stores blood test information as described above.

In a particular embodiment, the database stores blood test information measured from a GGE-based test, taken alone or combined with other blood tests. Such tests are described in U.S. Pat. No. 5,925,229, entitled“Low density lipoprotein fraction assay for cardiac disease risk” and U.S. Pat. No. 6,576,471, entitled“Methods, systems, and computer program products for analyzing and presenting NMR lipoprotein-based risk assessment results” , the contents of which are incorporated herein by reference.

In typical embodiments, the LIMS is hosted at a first location (e.g., in a laboratory that performs the blood tests), and the database is hosted at a second location (e.g., a data center). In this case, the first and second locations typically connect to each other through the Internet. The Internet-based system typically features a website that displays the blood test, vital sign, exercise, and personal information. In embodiments, the website includes a first web interface that displays information for a single patient, and a second web interface that displays information for a group of patients. For example, the second web interface could be used by a physician or clinical educator associated with a number of patients. Both web interfaces typically include multiple web pages that, in turn, feature both static and dynamic content, described in detail below.

The monitoring device typically measures: 1) heart rate; 2) systolic, diastolic, and pulse blood pressure; 3) pulse oximetry; and 4) cardiac ‘waveforms’ that can be further processed to determine arrhythmias, blood pressure load, and other cardiac properties. These properties can be measured daily as a one-time measurement, or quasi-continuously (e.g., every 30 seconds) during exercise. Preferably the monitoring device measures blood pressure without using a cuff in a matter of seconds, meaning patients can quickly and easily monitor this and other vital signs with minimal discomfort. This means patients can easily measure their vital signs throughout the day (e.g., while at work), thereby generating a complete set of information, rather than just a single, isolated measurement. In addition, the monitoring device can collect weight and percent body fat from a bathroom scale (using, e.g., a wired or wireless link), and exercise-related properties, such as steps (using an internal pedometer circuit), calories burned (using sensor inputs and associated algorithms), and exercise time (using a simple clock).

In other embodiments, the monitoring device includes an interface (e.g., an RS232 serial port, USB serial port, or wireless interface) to a personal computer. The wireless interface can include protocols such as Bluetooth™, 802.11, 802.15.4, and part-15. Typically, in this embodiment, the Internet-based system includes a software program that, when launched, collects vital sign and exercise information from the monitoring device. In response, software components associated with the Internet-based system process the blood test, vital sign, exercise, and personal information to generate personalized content for the patient. For example, the personalized content may include a report describing this information, personalized recommendations for diet and exercise, or reference articles. Similarly, the Internet-based system may link to ‘chat rooms’ or internal email systems that allow patients to communicate with one another.

The invention has many advantages, particularly because it provides a system that processes real-time information to, among other things, help a patient comply with a personalized cardiovascular risk reduction program. The program analyses blood test, vital sign, exercise, and personal information, taken alone or combined, to generate customized, patient-specific programs that can be quickly updated and modified. The program then provides personalized programs and their associated content to the patient through a messaging platform that sends information to a website, email address, wireless device, or monitoring device. Ultimately the Internet-based system, monitoring device, and messaging platform combine to form an interconnected, easy-to-use tool that can engage the patient in a disease-management program, encourage follow-on medical appointments, and build patient compliance. These factors, in turn, can help the patient lower their risk for certain medical conditions, such as CVD.

These and other advantages of the invention will be apparent from the following detailed description and from the claims.

### BRIEF DESCRIPTION OF THE DRAWINGS

FIG. 1 is a high-level schematic view of an Internet-based system that collects and analyzes blood test information from a blood test and vital sign and exercise information from a monitoring device;

FIG. 2 is a detailed schematic view of software and hardware components associated with the Internet-based system of FIG. 1;

FIG. 3A is a semi-schematic view of the monitoring device of FIG. 1 that measures blood pressure, pulse oximetry, heart rate, glucose levels, weight, and steps traveled;

FIG. 3B is a semi-schematic view of the monitoring device of FIG. 3A worn on a patient's belt;

FIG. 4 is a semi-schematic view of the monitoring device of FIGS. 3A and 3B connecting through a USB port to either a personal computer or a PDA; and, FIG. 5 is a schematic diagram of the electrical components of the monitoring device of FIGS. 3A and 3B.

### DETAILED DESCRIPTION OF THE INVENTION

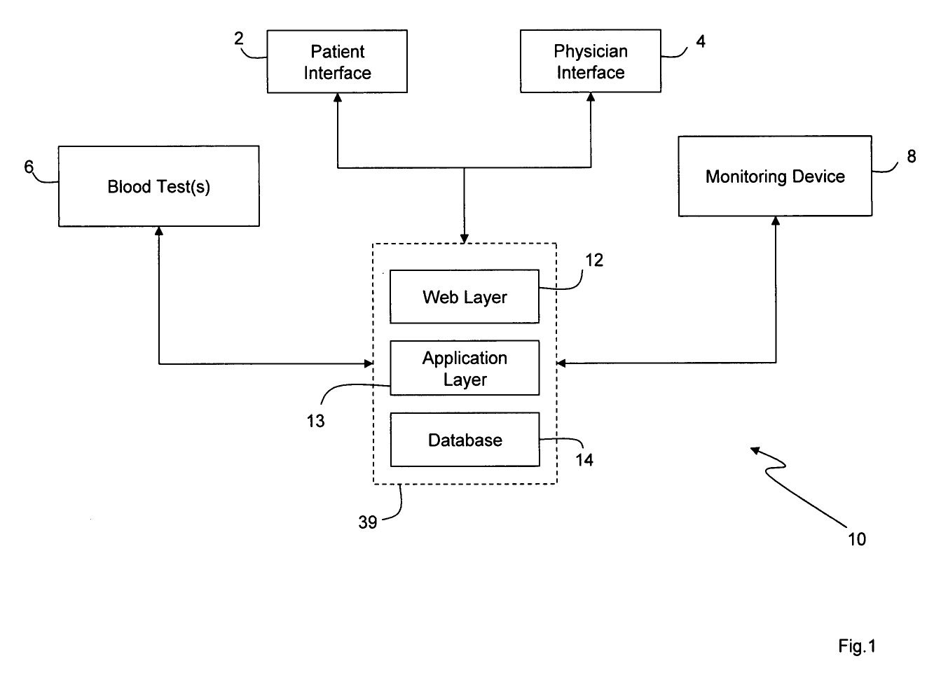

FIG. 1 shows an Internet-based system 10 according to the invention that collects blood test information from one or more blood tests 6, and vital sign and exercise information from a monitoring device 8. The Internet-based system 10 features a web application 39 that manages software for a database layer 14, application layer 13, and interface layer 12 for, respectively, storing, processing, and displaying information. The web application 39 renders information from a single patient on a patient interface 2, and information from a group of patients on a physician interface 4. More specifically, within the web application 39, the application layer 13 features information-processing algorithms that analyze the blood test, vital sign, and exercise information stored in the database layer 14. Analysis of this information can yield a metabolic and cardiovascular risk profile that, in turn, can help the patient comply with a cardiovascular risk reduction program. Specifically, based on this analysis, the interface layer 12 may render one or more web pages that describe a personalized program that includes reports and recommendations for diet, exercise, and lifestyle, along with content such as recipes and news and reference articles. These web pages are available on both the patient 2 and physician 4 interfaces.

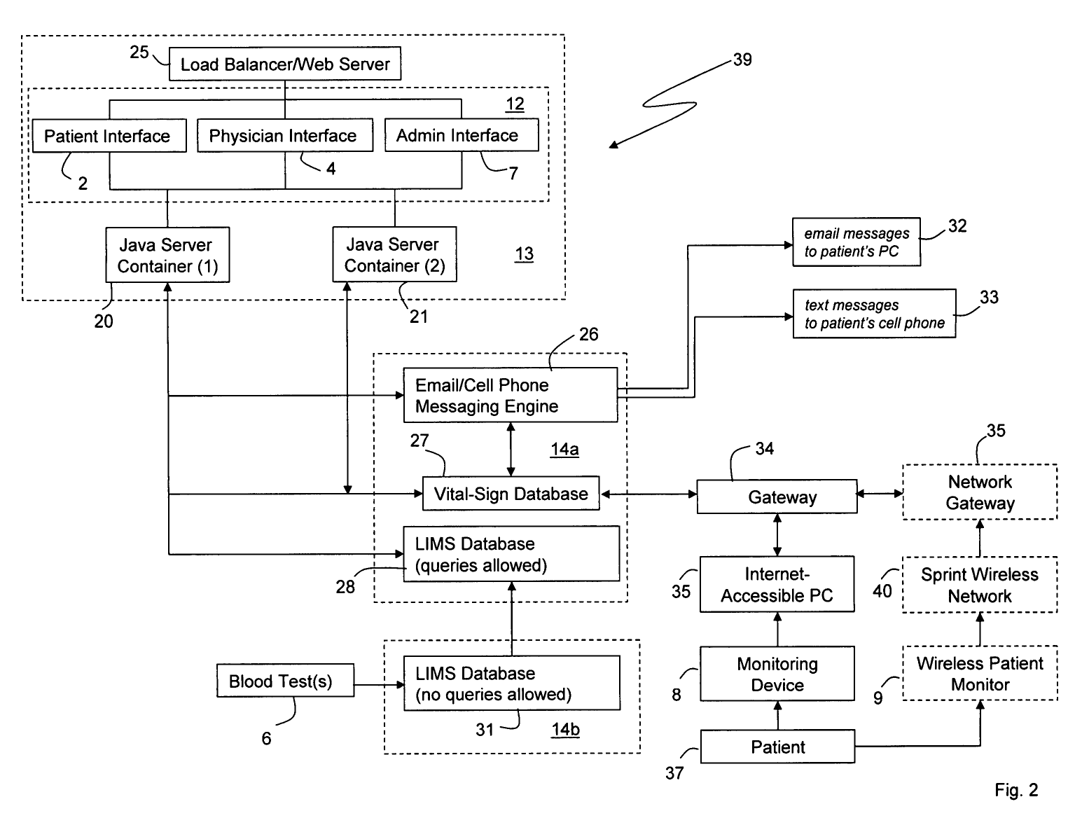

FIG. 2 provides a more detailed view of the web application 39. The database layer 14a, 14b features a LIMS database 31, which automatically receives information directly from a blood test (e.g., an GGE-based test), and is typically located proximal to laboratory equipment used for the test. Typically no external database queries are performed on the LIMS database 31; its function is simply to store diagnostic information for each test. The LIMS database 31 periodically (e.g., once every 24 hours) uploads information to a matched LIMS database 28 hosted at an external site, e.g. a data center. Also hosted at the data center is a vital sign database 27 that collects vital sign information from a monitoring device 8 associated with a patient 37. During operation, the monitoring device 8 measures vital sign and exercise information from the patient as described in more detail below. The patient 37 then plugs the monitor 8 into a USB port on an Internet-accessible personal computer 35, and logs into the patient interface 2. In response the patient interface 2 launches a software program that collects vital sign and exercise information from the monitor 8 and sends it to an Internet-based gateway software program 34. The gateway software program 34 formats the information and then sends it to the vital sign data database 27, where it is stored in memory for later processing.

The web application 39 additionally includes an administrative interface 7 that allows a user to, e.g., maintain and modify the application, query the database, and perform other administrative functions. The application 39 also includes a software load balancer/web server 25 that processes incoming http/https requests to regulate load placed on the site by outside users.

First 20 and second 21 Java server containers run software algorithms that process blood test information from the matched LIMS database 28 and vital sign information from the vital sign database 27. For example, the Java server containers 20, 21 may run a software algorithm that analyzes trends in the patient's vital sign information (e.g., their weight, resting heart rate, and blood pressure) and blood test information. In response, the algorithm can generate a personalized program that suggests a personalized diet or exercise routine. This personalized program is typically conveyed to both the patient 37 and their physician through the patient 2 and physician 4 interfaces. In addition, the web application 39 includes a bidirectional email/cell phone messaging engine 26 that sends reminders and feedback associated with the program in the form of emails 32 sent to the patient's computer, or text messages 33 sent to the patient's cell phone. The messaging engine 26 can also receive information for the patient, e.g. a confirmation of an appointment.

In related applications, a third party, such as a physician, clinical educator, physician, nurse, nurse practioners, dietician, physical therapist, personal trainer, or other professional with access to the Internet-based system, may use the Internet-based system to convey information to the patient. This can be done through the messaging engine 26 described above, or through a telephone call, on-line demonstration, or face-to-face meeting.

The web application 39 can also accept incoming vital sign information from a wireless patient monitor 9, such as that described in CUFFLESS BLOOD PRESSURE MONITOR AND ACCOMPANYING WIRELESS MOBILE MONITOR (U.S. Ser. No. 10/967,511; filed Oct. 18, 2004), the contents of which are incorporated by reference. In this embodiment, the wireless patient monitor 9 measures vital sign information from the patient 37, and transmits this information through a wireless network 40 to a network gateway software system 35. Using an Internet protocol, this system 35 sends the information to the gateway software system 34, which then stores it in the vital sign database 27 as described above.

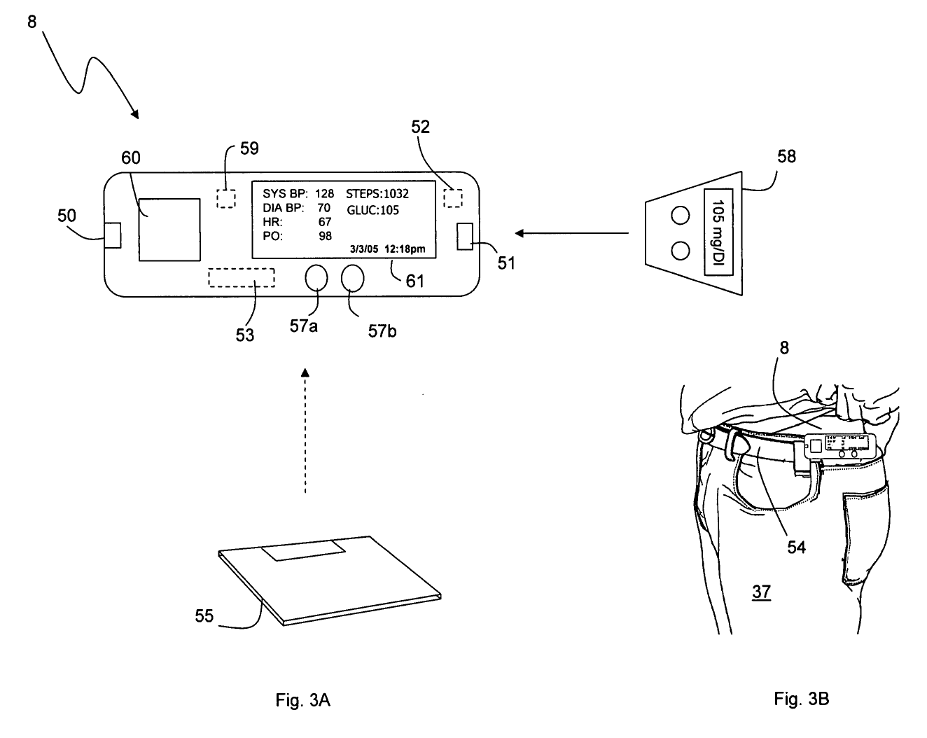

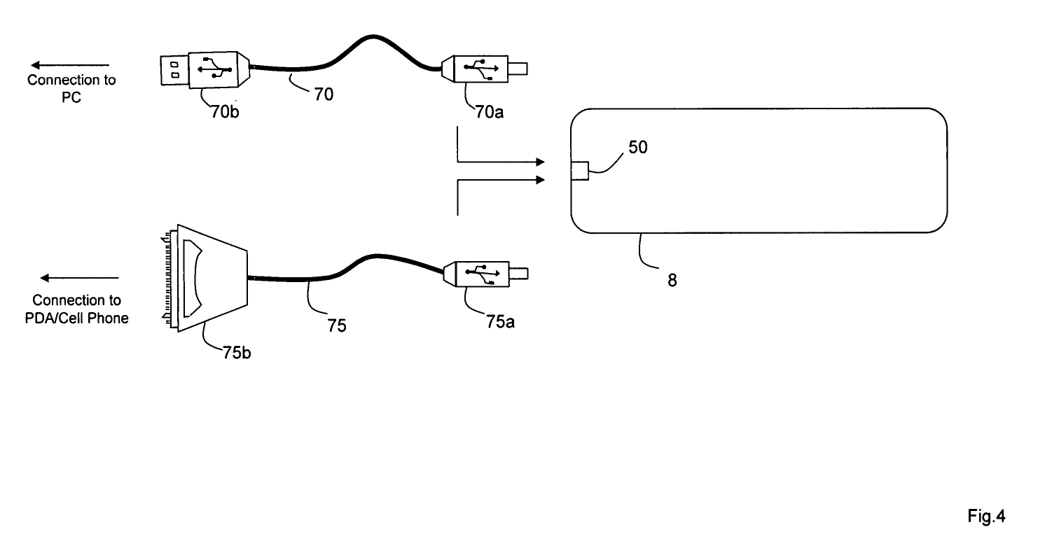

FIGS. 3A and 3B show a small-scale, vital sign monitoring device 8 according to the invention that measures information such as blood pressure, pulse oximetry, heart rate, glucose levels, calories burned, steps traveled, and dietary information from a patient 37 as described above. The monitoring device 8, typically worn on the patient's belt 54, features: i) an integrated, optical ‘pad sensor’ 60 that cufflessly measures blood pressure, pulse oximetry, and heart rate from a patient's finger as described in more detail below; and ii) an integrated pedometer circuit 52 and array of heat sensors 59 that measure, respectively, steps and calories burned. To receive information from external devices, the monitoring device 8 also includes: i) a mini USB connector 50; ii) a serial connector 51 that connects and downloads information from an external glucometer 58; and iii) a short-range wireless transceiver 53 that receives information such as body weight and percentage of body fat from an external scale 55. The patient views information from a display 61 (e.g. an LCD), and can interact with the device 8 (e.g., select a feature; reset or reprogram it) using a series of buttons 57a, 57b.

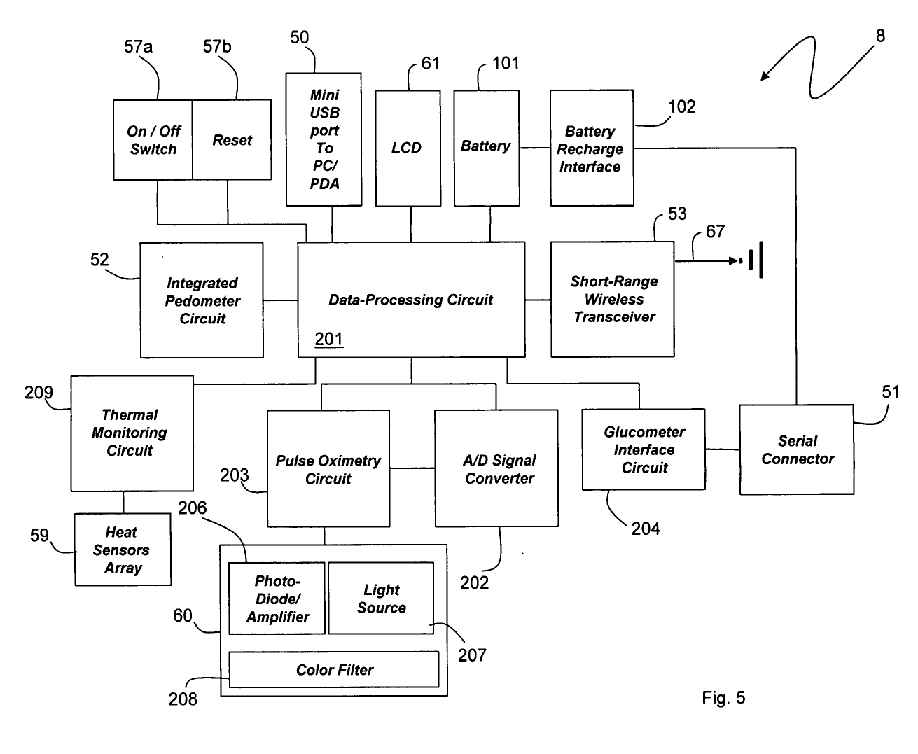

Referring to FIG. 4, the monitoring device 8 can transfer information to the Internet-based system through the mini USB port 50. The USB port 50 can connect to an external personal computer through a first cable 70 terminated with a conventional USB connector 70b. Alternatively, the monitoring device connects to a PDA through a second cable 75 terminated with a serial connector 75b. The PDA, for example, can be a conventional wireless device, such as a cellular phone.

FIG. 5 shows a preferred embodiment of the electronic components within the monitoring device 8. A data-processing circuit 201 controls: i) a pulse oximetry circuit 203 connected to the optical pad sensor 60; ii) LCD 61; iii) a glucometer interface circuit 204 that connects to an external glucometer through a serial connector 51; iv) an integrated pedometer circuit 52; v) a thermal-monitoring circuit 209 that processes information from an array of heat sensors 59; and vi) a short-range wireless transceiver 53. During operation, the optical pad sensor 60 generates an optical waveform that the data-processing circuit 201 processes to measure blood pressure, pulse oximetry, and heart rate as described in more detail below. The sensor 60 combines a photodiode/amplifier 206, color filter 208, and light source 207 on a single silicon-based chip. The light source 207 typically includes light-emitting diodes that generate both red (λ˜350 nm) and infrared (λ˜1050 nm) radiation.

As the heart pumps blood through the patient's finger, blood cells absorb and transmit varying amounts of the red and infrared radiation depending on how much oxygen binds to the cells' hemoglobin. The photodiode/amplifier 206 detects and amplifies transmission at both red and infrared wavelengths, and in response generates a radiation-induced current that travels through the sensor 60 to the pulse-oximetry circuit 203. The pulse-oximetry circuit 203 connects to an analog-to-digital signal converter 202, which converts the radiation-induced current into a time-dependent optical waveform. The analog-to-digital signal converter 202 sends the optical waveform to the data-processing circuit 201 that processes it to determine blood pressure, pulse-oximetry, and heart rate, which are then displayed on the LCD 61. Once information is collected, the monitoring device 8 can send it through the mini USB port 50 to a personal computer or PDA.

In embodiments, the monitoring device 8 can processes information from an integrated pedometer circuit 52 to measure steps. Such a circuit is described in U.S. Pat. No. 6,473,483, entitled ‘Pedometer’, the contents of which are incorporated herein by reference. Likewise, the heat sensor array 59 coupled to the thermal monitoring circuit 209 measures heat given off by the patient during exercises and day-to-day activities. This information can then be used to calculate calories burned or simply the user's temperature. Such a circuit is described in U.S. Pat. No. 6,595,929, entitled ‘System for monitoring health, wellness and fitness having a method and apparatus for improved measurement of heat flow’ the contents of which are incorporated herein by reference. In other embodiments, the monitoring device 8 connects through the serial connector 51 and glucometer interface circuit 204 to an external glucometer to download blood-glucose levels.

In still other embodiments, the monitoring device 8 uses heart rate, as calculated by the optical pad sensor 60, to calculate calories burned. Such a calculation is described in U.S. Pat. No. 6,013,009, “Walking/Running Heart Rate Monitoring System,” the contents of which are incorporated here in by reference. The monitoring device can also include a global positioning system (GPS) that measures the patient's location or distance traveled (e.g., during exercise).

The monitoring device 8 can include a short-range wireless transceiver 53 that sends information through an antenna 67 to a matched transceiver embedded in an external monitor, e.g. a personal computer or PDA. The short-range wireless transceiver 53 can also receive information, such as weight and body-fat percentage, from an external scale, as described with reference to FIG. 3A. A battery 101 powers all the electrical components within the monitoring device 8, and is preferably a metal hydride battery (generating 3-7V) that can be recharged through a battery-recharge interface 102. The battery-recharge interface 102 can receive power through the mini USB port 50. Buttons control functions within the monitoring device such as an on/off switch 57a and a system reset 57b.

In other embodiments, the pad sensor can also include an electrode, coupled to a reference electrode, which detects an electrical impulse from the patient's skin that is generated each time the patient's heart beats. Following a heartbeat, the electrical impulse travels essentially instantaneously from the patient's heart to the pad sensor, where the electrode detects it relative to the reference electrode to generate an electrical waveform. At a later time, a pressure wave induced by the same heartbeat propagates through the patient's arteries and arrives at the pad sensor, where the light source/amplifier and photodiode detect it as described above to generate the optical waveform. The propagation time of the electrical impulse is independent of blood pressure, whereas the propagation time of the pressure wave depends strongly on pressure, as well as mechanical properties of the patient's arteries (e.g., arterial size, stiffness). The data-processing circuit runs an algorithm that analyzes the time difference (ΔT) between the arrivals of these signals, i.e. the relative occurrence of the optical and electrical waveforms as measured by the pad sensor. Calibrating the measurement (e.g., with a conventional blood pressure cuff) accounts for patient-to-patient variations in arterial properties, and correlates ΔT to both systolic and diastolic blood pressure. This results in a calibration table. During an actual measurement, the calibration source is removed, and the data-processing circuit analyzes ΔT along with other properties of the optical and electrical waveforms and the calibration table to calculate the patient's real-time blood pressure.

Methods for processing optical and electrical waveforms to determine blood pressure without using a cuff are described in the following co-pending patent applications, the entire contents of which are incorporated by reference: 1) CUFFLESS BLOOD-PRESSURE MONITOR AND ACCOMPANYING WIRELESS, INTERNET-BASED SYSTEM (U.S. Ser. No. 10/709,015; filed Apr. 7, 2004); 2) CUFFLESS SYSTEM FOR MEASURING BLOOD PRESSURE (U.S. Ser. No. 10/709,014; filed Apr. 7, 2004); 3) CUFFLESS BLOOD PRESSURE MONITOR AND ACCOMPANYING WEB SERVICES INTERFACE (U.S. Ser. No. 10/810,237; filed Mar. 26, 2004); 4) VITAL SIGN MONITOR FOR ATHLETIC APPLICATIONS (U.S. Ser. No. ; filed Sep. 13, 2004); 5) BLOOD PRESSURE MONITORING MONITOR FEATURING A CALIBRATION-BASED ANALYSIS (U.S. Ser. No. 10/967,610; filed Oct. 18, 2004); 6) PERSONAL COMPUTER-BASED VITAL SIGN MONITOR (U.S. Ser. No. 10/906,342; filed Feb. 15,2005); 7) PATCH SENSOR FOR MEASURING BLOOD PRESSURE WITHOUT A CUFF (U.S. Ser. No. 10/906,315; filed Feb. 14, 2005); and 8) SMALL-SCALE, VITAL SIGNS MONITORING MONITOR, SYSTEM AND METHOD (U.S. Ser. No. 10/907,440; filed Mar. 31, 2005).

Other embodiments are also within the scope of the invention. In particular, the web pages used to display information can take many different forms, as can the manner in which the data are displayed. Web pages are typically written in a computer language such as ‘HTML’ (hypertext mark-up language), and may also contain computer code written in languages such as Java and Java script for performing certain functions (e.g., sorting of names). The web pages are also associated with database software (provided by companies such as Oracle and Microsoft) that is used to store and access data. Equivalent versions of these computer languages and software can also be used. In general, the graphical content and functionality of the web pages may vary substantially from what is shown in the above-described figures. In addition, web pages may also be formatted using standard wireless access protocols (WAP) so that they can be accessed using wireless devices such as cellular telephones, PDAs, and related devices.

Different web pages may be designed and accessed depending on the end-user. As described above, individual users have access to web pages that only their vital sign data (i.e., the patient interface), while organizations that support a large number of patients (e.g. hospitals) have access to web pages that contain data from a group of patients (i.e., the physician interface). Other interfaces can also be used with the web site, such as interfaces used for: hospitals, insurance companies, members of a particular company, clinical trials for pharmaceutical companies, and e-commerce purposes. Vital sign information displayed on these web pages, for example, can be sorted and analyzed depending on the patient's medical history, age, sex, medical condition, and geographic location.

The web pages also support a wide range of algorithms that can be used to analyze data once it is extracted from the data packets. For example, the above-mentioned text message or email can be sent out as an ‘alert’ in response to vital sign or blood test information indicating a medical condition that requires immediate attention. Alternatively, the message could be sent out when a data parameter (e.g. blood pressure, heart rate) exceeded a predetermined value. In some cases, multiple parameters can be analyzed simultaneously to generate an alert message. In general, an alert message can be sent out after analyzing one or more data parameters using any type of algorithm. These algorithms range from the relatively simple (e.g., comparing blood pressure to a recommended value) to the complex (e.g., predictive medical diagnoses using ‘data mining’ techniques). In some cases data may be ‘fit’ using algorithms such as a linear or non-linear least-squares fitting algorithm. In general, any algorithm that processes data collected with the above-described method is within the scope of the invention.

In certain embodiments, the above-described can be used to characterize a wide range of maladies, such as diabetes, heart disease, congestive heart failure, sleep apnea and other sleep disorders, asthma, heart attack and other cardiac conditions, stroke, Alzheimer's disease, and hypertension.

Still other embodiments are within the scope of the following claims.

I claim as my invention:

1. A system for monitoring a patient, comprising:
a database that stores the patient's blood test information;
a monitoring device comprising systems that monitor the patient's vital sign and exercise information;
a database that receives vital sign and exercise information from the monitoring device; and
an Internet-based system configured to receive, store, and display the blood test, vital sign, and exercise information.

2. The system of claim 1, further comprising a database configured to store information describing the patient's cholesterol.

3. The system of claim 2, wherein the database is further configured to store a distribution of the patient's LDL cholesterol.

4. The system of claim 2, wherein the database is further configured to store a distribution of the patient's HDL cholesterol.

5. The system of claim 1, wherein the first database is further configured to store information describing at least one of the following properties: C-reactive protein, Apoliprotein A-1, Apoliprotein B, Apoliprotein E Isoforms, Fibrinogen, Folate, HbA_{1c}, Glucose, Insulin, Homocysteine, Lipoprotein (a), and Chlamydia.

6. The system of claim 1, wherein the database that stores the patient's blood test information is a laboratory information monitoring system.

7. The system of claim 6, wherein the laboratory information monitoring system is hosted at a first site, and the database that receives vital sign and exercise information from the monitoring device is hosted at a second site.

8. The system of claim 7, wherein the first and second sites connect to each other through an Internet connection.

9. The system of claim 1, wherein the Internet-based system comprises a website to display the blood test, vital sign and exercise information.

10. The system of claim 9, wherein the website comprises a first web interface that displays information for a single patient, and a second web interface that displays information for a group of patients.

11. The system of claim 10, wherein the first web interface comprises at least one web page that displays the patient's blood test information, at least one web page that displays the patient's vital sign information, and at least one web page that displays the patient's exercise information.

12. The system of claim 1, wherein the monitoring device further comprises an interface to a personal computer.

13. The system of claim 12, wherein the interface to the personal computer is a serial interface.

14. The system of claim 13, wherein the interface to the personal computer is a wireless interface.

15. The system of claim 15, wherein the wireless interface is based on a protocol selected from the following: Bluetooth™, 802.11, 802.15.4, and part-15.

16. The system of claim 12, wherein the Internet-based system further comprises a software program that, when launched, collects vital sign and exercise information from the monitoring device.

17. The system of claim 1, wherein the Internet-based system further comprises a software component that processes the blood test and vital sign information to generate personalized content for the patient.

18. The system of claim 17, wherein the personalized content is comprised by a report.

19. The system of claim 18, wherein the report comprises personalized recommendations on diet and exercise.

20. The system of claim 1, wherein the Internet-based system further comprises a software component that processes at least one of the vital sign and exercise information to generate a personalized email message for the patient.

21. The system of claim 1, wherein the Internet-based system further comprises a software component that processes at least one of the vital sign and exercise information to generate a personalized text message for the patient.

22. The system of claim 21, wherein the personalized text message is sent to a wireless device.

23. The system of claim 22, further configured to send a personalized text message to cellular telephone or wireless personal digital assistant.

24. The system of claim 1, wherein the vital sign information comprises information describing at least one of heart rate, blood pressure, pulse oximetry.

25. The system of claim 1, wherein the exercise information comprises at least one of steps, calories burned, and exercise time.

26. The system of claim 1, wherein the monitoring device is further configured to monitor weight information.

27. The system of claim 26, wherein the weight information comprises weight and percent body fat.

28. The system of claim 27, wherein the monitoring device further comprises an interface to a weighing scale.

29. The system of claim 28, wherein the interface to the weighing scale is a wireless interface.
